(19) United States
(12) Patent Application Publication
(10) Pub. No.: US 2015/0216718 A1
(43) Pub. Date: Aug. 6, 2015

(54) DEVICES, SYSTEMS AND METHODS FOR THERMOELECTRIC HEATING AND COOLING OF MAMMALIAN TISSUE

(71) Applicant: BOARD OF REGENTS, THE UNIVERSITY OF TEXAS SYSTEM, Austin, TX (US)

(72) Inventors: Kenneth R. Diller, Elgin, TX (US); Daniel W. Hensley, Emeryville, CA (US); Andrew E. Mark, Austin, TX (US); Sepideh Khoshnevis, Austin, TX (US)

(21) Appl. No.: 14/419,269

(22) PCT Filed: Aug. 5, 2013
(86) PCT No.: PCT/US2013/053611

§ 371 Date: Feb. 3, 2015

Related U.S. Application Data
(60) Provisional application No. 61/679,438, filed on Aug. 3, 2012.

(51) Int. Cl. A61F 7/00

(52) U.S. Cl. CPC A61F 7/007; A61F 2007/0095

(57) ABSTRACT

Provided are devices for heating, cooling, or maintaining temperature of mammalian tissue. Also provided are methods for using the same. The devices optionally include a plurality of thermoelectric modules. The modules are selectively operable in temporal and spatial dimensions.

### FIELD OF THE INVENTION

The present disclosure relates generally to devices, systems and methods for thermoelectric heating and cooling of mammalian tissue.

### BACKGROUND

In a wide range of settings, both clinical and those involving personal comfort, cooling and heating effects are applied to mammalian tissue or skin. The application of heating or cooling effects to the mammalian tissue or skin can provide therapeutic treatment of tissues.

For example, the application of heating or cooling effects can relieve pain, relax muscles, alter blood vessels, and relax connective tissue. By applying heating or cooling effects or various stimuli, the core temperature of the mammalian body may be lowered, raised, or otherwise maintained. Furthermore, the application of a hot or cold source may be beneficial in the post-operation setting. Following muscle, tendon, ligament, and other soft tissue damage, applications of heat and/or cold may be beneficial for controlling blood flow, swelling, inflammation, pain, metabolism, and general healing processes. Muscle, tendon, and ligament damage may cause these tissues to undergo shortening, usually due to guarded joint positioning from pain, swelling, and muscle spasm. In most rehabilitative efforts, the goal is aimed at gradual restoration of joint motion via stretching and gentle range of motion exercises. Application of a heating effect, for example, may facilitate the lengthening of collagen tissues within these structures, thereby aiding in the stretching and rehabilitative processes.

In certain circumstances, the application of devices or systems that provide a heating or cooling effect to mammalian tissue or skin is difficult because of the contours of the mammalian body. Furthermore, accurate control of the temperature of the heating or cooling effect may be difficult, and in some instances it may be desirable for a thermal device to be conveniently portable.

### SUMMARY

The present application relates to systems and methods for thermoelectric heating and cooling of mammalian tissue. For example, the systems and methods can be used to cool or warm mammalian tissue with thermoelectric (TE) modules.

Provided herein are devices for heating, cooling, or maintaining temperature of mammalian tissue. The devices include one or more thermoelectric modules, each module having an upper and a lower surface. At least two surfaces are selectively operable to reject heat from one of the upper or lower surfaces and to absorb heat from the opposing upper and lower surfaces.

Optionally, two or more thermoelectric modules are positioned relative to each other in a predetermined spatial orientation. Optionally, each module of the plurality is positioned relative to the other modules of the plurality in a predetermined spatial orientation.

Example devices further include at least one control unit that is in operable communication with each thermoelectric module. The control unit optionally operates to regulate flow of electric current through one or more of the thermoelectric modules. The devices optionally further include a plurality of conductors and each thermoelectric module is operatively connected to a pair of the conductors. Regulation of electrical current flow through a given thermoelectric module optionally comprises establishing a voltage differential between the pair of conductors that is in operative communication with that thermoelectric module.

Optionally, the electric current flow through a given module causes heat rejection by one of the upper or lower surface of the module and heat absorption at the other of the upper or lower surface of the module. Optionally, the lower surface is proximate to mammalian tissue relative to the upper surface and the lower surface absorbs heat and the upper surface rejects heat. The lower surface optionally absorbs heat from the mammalian tissue and the mammalian tissue is optionally cooled or its temperature maintained. Optionally, the lower surface is proximate the mammalian tissue relative to the upper surface and the lower surface rejects heat and the upper surface absorbs heat. In this case, the lower surface optionally rejects heat into the mammalian tissue and the mammalian tissue is heated or its temperature maintained.

The example devices optionally further include a connecting substrate and at least two thermoelectric modules that are fixed to the substrate. The fixation to the substrate orients the position of at least two thermoelectric modules relative to each other. Optionally, the connecting substrate is flexible. Optionally, at least a portion of the flexible substrate and at least two thermoelectric modules are conformable to a portion of the mammal anatomy or tissue. Optionally, the orientation of one or more thermoelectric modules are adjustable in three dimensions. Optionally, the connecting substrate is thermally conductive. Optionally, the connecting substrate has a low force deformability. Optionally, each of the thermoelectric modules are fixed to the substrate to orient their position relative to each other.

Activation of the thermoelectric modules can be varied in the temporal and spatial dimensions. For example, at least two of the thermoelectric modules are selectively operable in a temporal dimension. In another example, each of the modules is selectively operable in a temporal dimension. At least two of the thermoelectric modules can also be selectively operable in a spatial dimension and these are also optionally selectively operable in the temporal dimension. In some examples, each of the modules is selectively operable in a spatial dimension. Optionally, the connecting substrate is a mesh.

In the example devices, at least two of the thermoelectric modules are positionable in overlying registration with the mammalian tissue. Optionally, the mammalian tissue is skin.

The devices optionally further include a barrier substrate positioned between the mammalian tissue and the thermoelectric modules. The barrier substrate is optionally disposable.

The example devices include thermoelectric modules that have a length dimension, a width dimension and a thickness dimension. The length dimension is optionally equal to the width dimension. The length dimension is optionally about 2.0 centimeters. In other examples, the length dimension is not equal to the width dimension.

In the example devices, each thermoelectric module is optionally spaced from each other thermoelectric module, and optionally, the spacing between the thermoelectric modules is the same.

The described devices further include a plurality of sensor units, wherein each sensor unit senses temperature proximate to at least one thermoelectric module. Optionally, each thermoelectric module has an associated sensor unit. Optionally, the devices include a control unit, having at least one processing device and a plurality of sensor units. Each thermoelectric module is associated with at least one of the sensor units and the sensor units sense temperature and communicate the sensed temperature to the control unit for processing. The sensor units are optionally positioned at the lower surface of one or more of the thermoelectric modules when the lower surface is more proximate the tissue than the upper surface. The sensor units optionally sense the temperature of the tissue or of the lower surface. The sensed temperature is optionally communicated to the control unit where it is used to adjust the operational characteristics of one or more thermoelectric module.

Also provided herein are temperature sources to drive heat transfer processes, wherein the temperature source is a thermoelectric (TE) material. For example, the sources are used to drive heat transfer processes in mammalian tissue. Optionally, the TE material is positioned at the site of treatment on a mammal. Optionally, the TE material is in thermal communication with mammal tissue without need for an intermediate convective transport medium. Optionally, the TE material is configured into a plurality of TE tiles each of which is individually and independently controllable.

The individual TE tiles are optionally mountable onto a flexible membrane that allows the aggregate TE tiles to conform to the shape of a mammalian surface to which they are applied. The flexible membrane optionally allows for differences in three-dimensional orientation between adjacent TE tiles. The flexible membrane optionally has properties of high thermal conductivity to provide thermal communication between a TE material and a mammalian tissue surface. Optionally, the flexible membrane has a low force elastic deformability to conform to the geometry of a mammalian surface to increase the contact area between the TE material and the mammalian surface. The flexible membrane is optionally tailored by addition of chemical constituents, tailoring of stoichiometry, or method of curing such that heat transfer, or other properties such as optical or electrical, may be varied.

Also provided is a flexible elastically deformable membrane, wherein the membrane increases the surface contact area between a curved mammalian surface and a contacting rigid TE material. The flexible membrane optionally has an open pore structure that can be loaded with a shear thinning gel. Optionally, the membrane has an open pore structure that can be loaded, injected, or impregnated with a fluid/material that affects the thermal, optical, electrical, or other properties in the manner of the membrane. Optionally the material is copper slurry or ultrasound gel.

Also provided is a control system for TE material tiles that enables temporal and spatial modulation of the temperature applied to the mammalian surface with feedback from individual thermal sensors associated with each tile. Optionally, all of the TE tiles are programmed to produce a positive heat flux. Optionally, all of the TE tiles are programmed to produce a negative heat flux. Optionally, the TE tiles are programmed to provide both positive and negative heat fluxes across the tiles in a checkerboard or similar fashion to illicit responses from the body's thermal receptors and therefore impact neural control and sensation of the affected tissues. Optionally, the TE tiles are controlled by individual PID feedback calculations based on sensors on the surface of the mammalian tissue and/or in the intervening material layers of the device. Optionally, the TE tiles are controlled by individual control feedback calculations based on temperature sensors at the surface of the mammalian tissue and/or in the intervening material layers of the device. Optionally, the TE tiles are controlled by individual control calculations based on heat flux sensors at the surface of the mammalian tissue and/or in the intervening material layers of the device. Optionally, the TE tiles are controlled by individual control calculations based on blood perfusion sensors at the surface of the mammalian tissue.

Also provided is a programmable control component that can provide and enforce a pattern of temperature modulation in time and position on the surface of a mammalian tissue. Also provided is a method of powering of a bioheat transfer device with only an electrical source. Also provided is a method of cooling the outer surface of a bioheat transfer device with a fan. Also provided is a method of cooling the outer surface of a bioheat transfer device with heat sink topologies, such as fins. Also provided is a method of cooling the outer surface of a bioheat transfer device with both heat sink topologies, such as fins, and fans. Also provided is a bioheat transfer device that consists of only a single physical unit. Also provided, is a bioheat transfer device that has no liquid flow tubes. Also provided is a switch to operate a TE material only when subject tissue is to be cooled or heated. Also provided is a TE material for application to a surface of mammalian tissue to cause a thermal load for substantially altering the temperature and energy stored in a tissue volume. Also provided is a TE material for application to or proximate to the surface of a mammalian tissue to cause an alteration to one or more local thermal afferent physiological signals that result in a predicted change of the thermal or thermoregulation function of the mammal.

Further provided is a TE device consisting of a plurality of TE tiles, wherein a tile is a single rigid, planar, and independently operable TE device. Optionally, the TE tiles are on the order of 1 cm². Optionally the device includes a plurality of independent TE tiles arranged in a parallel array. Optionally, the array exists as a rigid plane. Optionally, the array is mounted on a flexible membrane. Optionally, the individual TE tiles are arranged with significant space between each other, but are attached to the flexible membrane in a manner so as they are freely moveable with respect to each other as the membrane moves. Optionally, the free movement is intended to surround a curved or highly irregular mammalian surface and produce a substantial degree of contact. Optionally, the individual TE tiles are arranged with simple hinge structures between adjacent tiles in addition to being attached to the flexible membrane in a manner so as they may move with respect to each other as the membrane moves. Optionally, the movement surrounds a curved or highly irregular mammalian surface and produces a substantial degree of contact while maintaining structural integrity.

The described TE devices can heat or cool dynamically with spatio-temporal resolution. Optionally, the individual TE tiles are bipolar or have a thermal polarity different by row, column, or in some pattern. Alternating cold/hot tiles optionally generates a unique or high-magnitude physiological response. Optionally, the physiological response could be an increase or decrease in blood flow in the underlying mammalian tissue or a pattern of time variation in blood flow.

In the described TE devices the flexible membrane optionally includes memory foam. Optionally, the flexible membrane provides a form factor and methods to modulate thermal properties of the device. This modulation is optionally achieved by altering membrane thickness. This modulation is optionally achieved via impregnation with gels of defined thermal, optical, or electrical properties. This modulation is optionally achieved by altering the porosity, density, specific heat, insulation, and heat diffusion anisotropy.

In the described devices the TE component is optionally reusable and an intervening flexible membrane is optionally disposable.

Also provided are methods of affecting temperature of mammalian tissue. The methods optionally include providing a device as described herein and positioning the device such that at least two of the thermoelectric modules are in overlying registration with the tissue with the lower surface proximate the tissue relative to the upper surface. An electric current flow is initiated through at least one of the thermoelectric modules to cause absorption of heat by the lower surface of the thermoelectric module from the mammalian tissue and rejection of heat by the upper surface of the thermoelectric module to cool the mammalian tissue.

The methods optionally further include initiating an electric current flow through at least one additional of the thermoelectric modules positioned in overlying registration with the tissue to cause absorption of heat by the lower surface of the thermoelectric module from the mammalian tissue and rejection of heat by the upper surface of the thermoelectric module to cool the mammalian tissue.

The methods optionally further include initiating an electric current flow through at least one additional of the thermoelectric modules positioned in overlying registration with the tissue to cause rejection of heat by the lower surface of the thermoelectric module into the mammalian tissue and absorption of heat by the upper surface of the thermoelectric module to heat the mammalian tissue.

Also provided are methods of affecting temperature of mammalian tissue that include providing a device as described herein and positioning the device such that at least two of the thermoelectric modules are in overlying registration with the tissue with the lower surface proximate the tissue relative to the upper surface. An electric current flow is initiated through at least one of the thermoelectric modules to cause rejection of heat by the lower surface of the thermoelectric module from the mammalian tissue and absorption of heat by the upper surface of the thermoelectric module to heat the mammalian tissue.

The methods optionally further include initiating an electric current flow through at least one additional of the thermoelectric modules positioned in overlying registration with the tissue to cause rejection of heat by the lower surface of the thermoelectric module from the mammalian tissue and absorption of heat by the upper surface of the thermoelectric module to heat the mammalian tissue.

The methods optionally further include initiating an electric current flow through at least one additional of the thermoelectric modules positioned in overlying registration with the tissue to cause absorption of heat by the lower surface of the thermoelectric module into the mammalian tissue and rejection of heat by the upper surface of the thermoelectric module to cool the mammalian tissue.

The methods described herein optionally include activating one or more thermoelectric module is selectively by initiating the current through that module at a predetermined time. The predetermined time for activation is optionally different for activation of one or more module. Optionally, the predetermined time of activation is based on feedback from mammalian tissue or from the mammal.

Also provided are methods of affecting temperature of mammalian tissue. The methods optionally include providing a device as described herein and positioning the device such that at least two of the thermoelectric modules are in overlying registration with the tissue with the lower surface proximate the tissue relative to the upper surface. An electric current flow is initiated through at least one of the thermoelectric modules to cause alternating absorption and rejection of heat by the lower surface of the thermoelectric module from the mammalian tissue and alternating rejection and absorption of heat by the upper surface of the thermoelectric module. The alternating rejection and absorption of heat enhances the healing process of the mammalian tissue by realizing the combined benefits of both heating and cooling.

Also provided are methods of affecting temperature of mammalian tissue. The methods optionally include providing a device as described herein and providing a battery power source to operate all of the device components, including all thermoelectric modules, cooling fans, control computers, and sensors. The complete device, including any power source, is portable and capable of operation without requiring connection to a power mains. All device components are packaged so that the device may be used portably in the field and applied to a subject while the subject is being transported manually or via a vehicle on air, land, or sea.

These and other features and advantages of the implementations of the present disclosure will become more readily apparent to those skilled in the art upon consideration of the following detailed description and accompanying drawings, which describe both the preferred and alternative implementations of the present disclosure.

### BRIEF DESCRIPTION OF THE DRAWINGS

In the drawings, like reference numbers and designations in the various drawings indicate like elements.

FIG. 1 is a schematic illustration of an example array of thermoelectric modules arranged on a flexible substrate and placed on a subject's knee;

FIG. 2 is a schematic illustration of an example array of thermoelectric modules;

FIG. 3 is a schematic illustration of an example thermoelectric module;

FIG. 4 is a schematic illustration of an example control unit;

FIG. 5 is a schematic illustration of an example thermoelectric module situated on the skin of a subject;

FIGS. 6 and 7 are flowcharts showing examples processes for applying a heating or cooling effect with a thermoelectric module;

FIG. 8A is a schematic illustration of an example bismuth telluride thermoelectric cooler;

FIG. 8B is a schematic illustration of an example heat sink and forced convection fan;

FIG. 9 is a graph showing the results from a test to determine temperatures associated with a thermoelectric module;

FIG. 10 is a graph showing the results from a test to determine temperatures associated with a thermoelectric module applied to skin surrogate material;

FIG. 11 is a graph showing the results from a test to determine temperatures associated with the cooling of human skin using a thermoelectric module;

FIG. 12 is a graph showing the results from a test to determine temperatures associated with the cooling of the skin surface of a human subject using a thermoelectric module; and

FIG. 13 is a graph showing the results from a test to determine the temperatures of the heating and cooling cycles of a thermoelectric module mounted on the skin of a human subject.

### DETAILED DESCRIPTION

Implementations of the present disclosure now will be described more fully hereinafter. Indeed, these implementations can be embodied in many different forms and should not be construed as limited to the implementations set forth herein; rather, these implementations are provided so that this disclosure will satisfy applicable legal requirements. As used in the specification, and in the appended claims, the singular forms “a,” “an,” and “the,” include plural referents unless the context clearly indicates otherwise. The term “comprising” and variations thereof as used herein is used synonymously with the term “including” and variations thereof and are open, non-limiting terms.

Mammalian body temperature is tightly regulated by an internal autonomic regulatory system which comprises central controllers and the blood circulatory system, plus mechanisms for adjusting the rate and locations of internal energy generation. The circulatory system covers the entire body and delivers heat from the body core to the peripheral areas, or, in less frequent circumstances, delivers heat from the periphery to the core.

Alteration of the blood flow through the skin plays an important role in temperature regulation. For example, nonglabrous skin vasodilation (dilation of arterioles and small arteries) and vasoconstriction (constriction of arterioles and small arteries) increase or decrease blood flow to match thermoregulatory needs. Both the processes of vasoconstriction and vasodilation are regulated by active means in response to a combination of local, systemic, and central inputs.

Normally, when body and/or environmental temperatures are high, vasodilation favors high blood flow to the surface areas involved with heat exchange, thus increasing heat loss to the environment and reduction in the deep body core region temperature. As environmental and/or body temperatures fall, vasoconstriction reduces blood flow to the skin surfaces and minimizes heat loss to the environment.

One important effector of the thermoregulatory system is controlled by blood flow to specialized skin areas of the body at non-hairy skin surfaces, also referred to as glabrous skin, and includes skin at the palms of the hands, soles of the feet, and the ears, cheeks, forehead, and nose regions or any area of skin that contains a special vascular structure that is effective in affecting heat transfer between circulating blood and the body surface. Basal to the skin in these areas are unique anatomical vascular structures called venous plexuses. These structures serve to deliver large volumes of blood adjacent to the skin surface under conditions of vasodilation. By this delivery of blood, significant heat transfer may occur for the maintenance of internal organs within a functional temperature range.

Blood is permitted to pass through the venous plexus structures by way of arteriovenous anastomoses (AVAs) that are blood vessels that directly shunt arterial blood to the veins without passing through the capillaries. When vasodilated, the AVAs have a diameter an order of magnitude or greater than the capillaries, thereby providing a low flow resistance pathway for blood circulating from the heart. At full vasodilation the AVAs present among the lowest flow resistance of the entire circulatory system, resulting in a considerable fraction of the total cardiac output flowing through them. The relative proximity of the cutaneous AVAs to the surface of the body, and the potential for carrying large rates of blood flow make the AVAs a very effective heat exchange vehicle in the thermoregulatory apparatus. The AVAs are an integral part of the body's heat transfer system, providing important thermoregulatory control. Regulation of the AVA flow diameter is unique in comparison to the cutaneous microcirculation in nonglabrous skin. Adjustment in the flow resistance through AVAs is controlled by activation and relaxation of vasoconstriction.

Thermal stimulation of the peripheral thermoregulatory control tissue can cause an increase in the mean value of AVA flow wherein the vasomotive fluctuations are superimposed at a higher average flow. Removal of the thermal stimulation of the peripheral thermoregulatory control tissue can result in a lowering of the mean AVA flow as the relaxing input to the AVA vasoconstriction activity is diminished. This direct coupling between thermal stimulation of peripheral thermoregulatory control tissue and AVA perfusion rate offers a powerful opportunity to manipulate convective heat transfer processes involving glabrous skin, including convective movement of energy between the body surface and core via the circulation of blood to and from the AVAs, without involving inputs from the thermoregulatory control tissue of the hypothalamus.

The operation of the thermoregulatory system is remarkably efficient over a broad spectrum of physiological states and environmental conditions. In certain circumstances, however, the thermoregulatory system is unable to maintain the core temperature within the set operational range, or there may be therapeutic or prophylactic reasons to override the system to cause changes in the core temperature beyond the normal range or to maintain a core temperature in the face of environmental or other conditions that may cause alterations of core body temperature. In these cases, devices and methods are applied interventionally either to assist or to override the thermoregulatory system to cool or warm core body temperature.

Surface cooling over large areas of skin is ineffective because it induces vasoconstriction, thereby eliminating direct circulation of blood between the skin and core, which is a much more effective heat transfer mechanism than parasitic conduction through the body structures. Alternatively, methods have been developed for cooling the core directly by infusion of large volumes of chilled saline solution into the circulatory system. Collateral drawbacks associated with this technology include the necessity of canulating the circulatory system under sterile conditions and introduction of added volume of fluid into the blood which can result in elevated blood pressure. Since this technology is typically practiced in a medical facility, precious time during the window of therapeutic opportunity (averaging about 90 minutes) is lost during transport following the precipitating event to a medical facility and the initiation of therapy.

Vasodilation of the vasoconstricted, or more mechanistically, relaxation of the vasoconstriction of AVAs, enables the resolution of foregoing thermal physiological challenges for alteration of mammalian core temperature.

Beyond the body core temperature context, in various clinical settings, it becomes appropriate or even necessary to apply heating or cooling effects to various parts of the body. For example, after a surgical procedure, medical professionals may recommend the application of a hot or cold source on the surgical site, in order to reduce pain, improve healing time, and/or reduce post-operation complications. In other cases, a person may apply a heating or cooling effect to various parts of the body for a therapeutic effect. For example, a person may experience pain in, on, or around the knee. Consequently, in order to reduce or ease the pain, a person may apply a hot or cold source to the knee area.

The present disclosure may further be applied for personal comfort, core temperature and thermoregulatory manipulation, and cryotherapy. For cryotherapy and therapeutic hypothermia, both of which are therapeutic in nature, the use of thermoelectric devices includes significant heat flow to the skin. Thermoregulatory manipulation involves control signals to the skin at a much smaller magnitude of heat transfer than is required for therapeutic applications. For example, thermoregulatory manipulation generally embodies a null zone of thermosensitivity around a thermal set point. As the control temperature varies within a small amount from the instantaneous set point, there are no regulatory effector signals generated until a threshold displacement from the set point is reached. Beyond the null zone, energy conservation or energy dissipation processes are activated.

There are natural variations in the set point associated with the circadian rhythm, but in some cases that set point is altered in ways that are undesirable. The hot flashes often experienced in conjunction with menopause fall into this category. A cooling source may be applied to achieve therapeutic effects. For example, an appropriately placed TE module may provide a cooling sensation on the skin to influence the status of thermal control tissues that will generate a signal to offset the heat dissipation processes that typify hot flash symptoms. The TE module may be switched on and off by a user as required to achieve relief from vasodilation and sweating for conditions not dictated by the energy state of the body.

Another sensory input use of the systems and devices disclosed herein includes a TE module for achieving the thermal grid effect. This phenomenon describes the threshold limit of physical separation at which the body is able to perceive the application of two adjacent thermal stimuli at different temperatures. An array of TE modules may optionally provide the heating and cooling conditions that define the ability to detect different temperatures, particularly in terms of the sensitivity to the difference in temperatures and physical separation of the stimulants. The fast response time and accurate control characteristics of the systems and devices described herein may allow measurement of the threshold of temporal sensitivity in modulation of applied temperature sources.

For the induction of therapeutic hypothermia, for example, thermoelectric materials may allow for control of core temperature. The physiological stress from the therapy is minimal and the process may function in coordination with natural thermoregulation processes. Other advantages may include minimal interference with parallel medical procedures.

In terms of cryotherapy, a TE module may afford the ability to modulate the temperature on the treatment site in a time-varying geometric pattern to maintain the overall value of cooling while avoiding long term ischemia at all locations. Use of one or more TE modules with alternating heating and cooling may be applied to enhance the healing of injured soft tissues.

TE refrigeration technology has been applied for medical therapeutic and personal comfort uses in the past, but in the context of locating the cooling source remotely from the skin surface to be cooled and using the convection of an intermediate heat transfer fluid to deliver the desired energy load to the human.

Many of the current devices and systems for providing cooling and heating effects to mammalian skin or tissue include a remote source of cooling or heating. For example, many devices and systems include a remote source of cooling or refrigeration, and these systems require the use of tethering and other means for the circulation of a cooling fluid from the remote source to the treatment site, and then back to the remote source for further refrigeration. These systems may also include a pumping mechanism, flow tubing, and remote heat sinks, in order to move thermal energy between a source or sink and the mammalian surface area. Thus, a need arises that allows the source of cooling or heating to be applied directly to the treatment site, as well as allows the temperature of the cooling or heating source to be varied both spatially and temporally.

The described devices, systems, and methods can be used to cool or heat the mammalian tissue through the use of thermoelectric materials. There have been significant advances in developing new thermoelectric materials that enable devices that use thermoelectric materials, such as for example the TE modules described herein, to operate at higher efficiencies than in the past. The present disclosure provides for thermoelectric modules overlaying the mammalian tissue or skin to produce a cooling or heating effect. Thus, instead of using a remote source of heating or cooling, which usually involves the circulation of a cooling fluid or heating fluid to the treatment site, the thermoelectric devices and systems disclosed and described in detail herein may be on or proximate to the skin or the treatment site.

As previously mentioned, currently available systems and devices may include a pumping mechanism, flow tubing, and remote heat sinks, in order to move thermal energy between a source or sink and the mammalian surface area. Furthermore, many of these systems and devices do not allow the adaptability of the heating and cooling effects to the contours of the mammalian body. However, the present disclosure optionally allows for a reduction in weight of the system due to the fewer number of components as compared to other devices and systems. The present disclosure also optionally provides for a smaller size device and system, which inevitably increases mobility and lower capital and operating costs. In addition to lower electrical power sourcing, the present disclosure may also reduce noise, vibration, and harshness (NVH).

The systems, devices, and methods disclosed herein may also provide for the accurate control of application temperature during various clinical settings, including thermal therapies. Furthermore, the present disclosure may eliminate the need for cumbersome hardware required to produce therapeutic cooling, as well as the limitations in being able to provide both heating and cooling cycles in a single device or system. Thus, the systems, methods, and devices disclosed herein may be used to drive local blood flows in surface tissues higher and lower by thermal input manipulations.

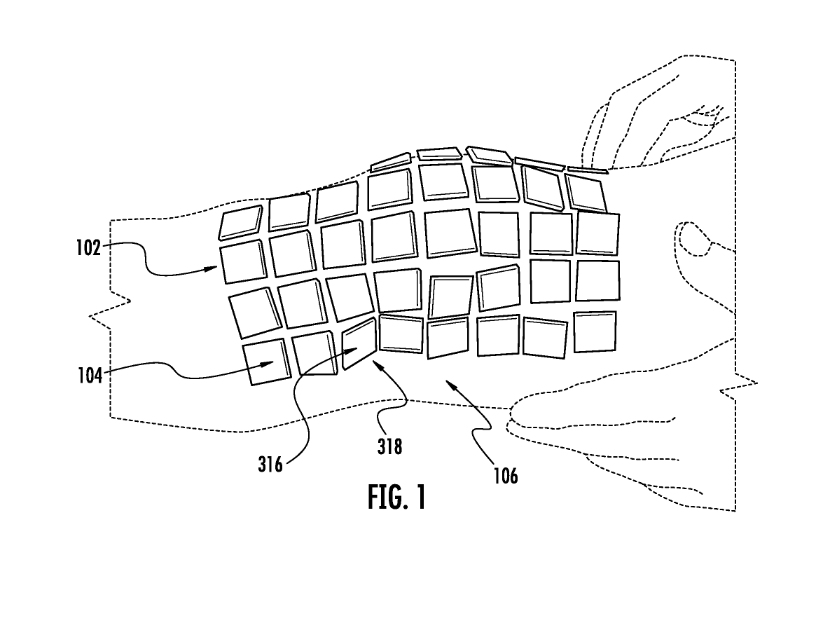

In these respects, provided herein are devices for heating, cooling, or maintaining temperature of mammalian tissue. Referring to FIG. 1, an example array 106 of thermoelectric modules are shown arranged on a flexible substrate and placed on a subject's knee. As shown in FIG. 1, an example device includes a plurality of TE modules 104. Each TE module 104 includes an upper surface 316 and a lower surface 318. At least two TE modules 104 are selectively operable to reject heat from one of the upper or lower surfaces 316 and 318 and to absorb heat one of the upper and lower surfaces 316 and 318.

Optionally, at least two of the TE modules 104 are positioned relative to each other in a predetermined spatial orientation, or array 106. Optionally, each module 104 of the plurality is positioned relative to the other modules 104 of the plurality in a predetermined spatial orientation.

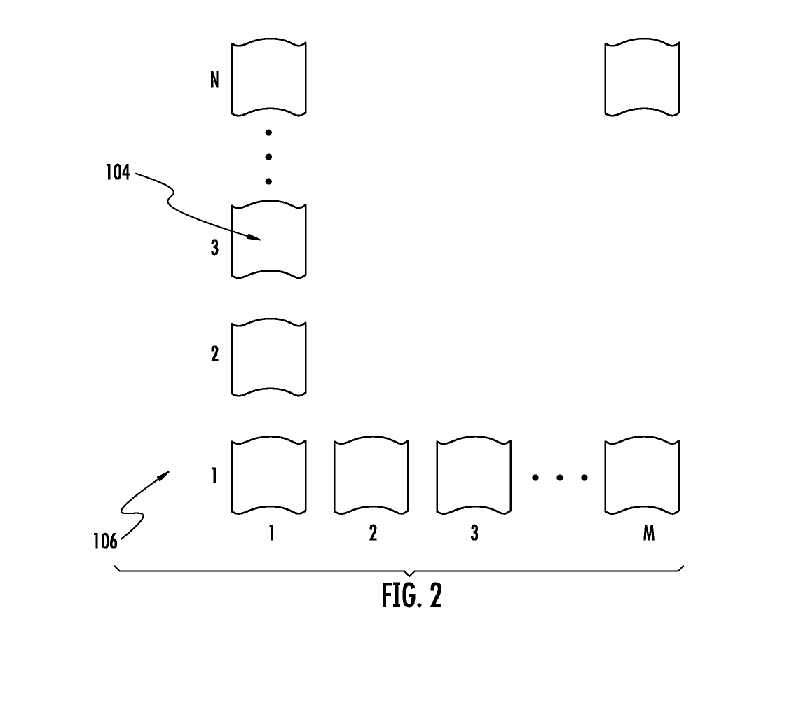

Referring to FIG. 2, an example predetermined spatial orientation of TE modules is shown. The predetermined spatial orientation may, for example, vary by m by n individual thermoelectric modules 104. The variables m and n may be adjusted to include as many or as few TE modules 104 as appropriate. The shape predetermined spatial orientation may include any shape that is appropriate for a situation or circumstances, and may be adapted accordingly. The orientation of the modules may be adjusted depending, for example, on the tissue to be cooled or heated, the extent of cooling or heating desired, the pattern, temporal or spatial, of cooling or heating desired.

Example devices further include at least one control unit 401 that is in operable communication with each thermoelectric module 104. Referring to FIG. 4, an example control unit is shown. The control unit 401 optionally operates to regulate flow of electric current through one or more of the thermoelectric modules 401. The devices optionally further include a plurality of conductors (302 and 304) and each thermoelectric module 104 is operatively connected to a pair of the conductors (302 and 304). Regulation of electrical current flow through a given thermoelectric module 104 optionally includes establishing a voltage differential between the pair of conductors (302 and 304) that is in operative communication with that thermoelectric module 104.

The control unit 401 includes power input 402 for providing electricity or power to the control unit 401. Optionally, the power input 402 may include battery or other appropriate power source, such that the control unit 401 and all of its components are optionally portable. Because of the portability and adaptability of the control unit 401 and all of its components, the control unit 401 and all of its components may be used to bring thermal therapies into the field for first responder use.

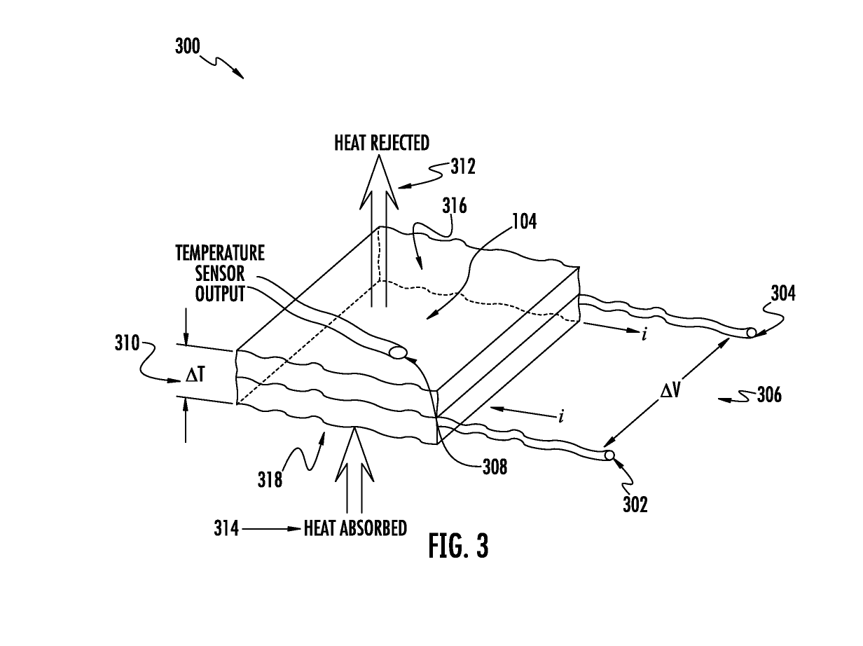

Referring to FIG. 3, an example thermoelectric module is shown. The electric current flow through a given module 104 optionally causes heat rejection 312 by one of the upper or lower surfaces 316 and 318 of the module 104 and heat absorption 314 at the other of the upper or lower surface 316 and 318 of the module 104. Optionally, the lower surface 318 is proximate the mammalian tissue relative to the upper surface 316 and the lower surface 318 absorbs heat and the upper surface 316 rejects heat. The lower surface 318 optionally absorbs heat from the mammalian tissue and the mammalian tissue is optionally cooled or its temperature maintained. Optionally, the lower surface 318 is proximate the mammalian tissue relative to the upper surface 316 and the lower surface 318 rejects heat into and the upper surface 316 absorbs heat. In this case, the lower surface 318 optionally rejects heat into the mammalian tissue and the mammalian tissue is heated or its temperature maintained.

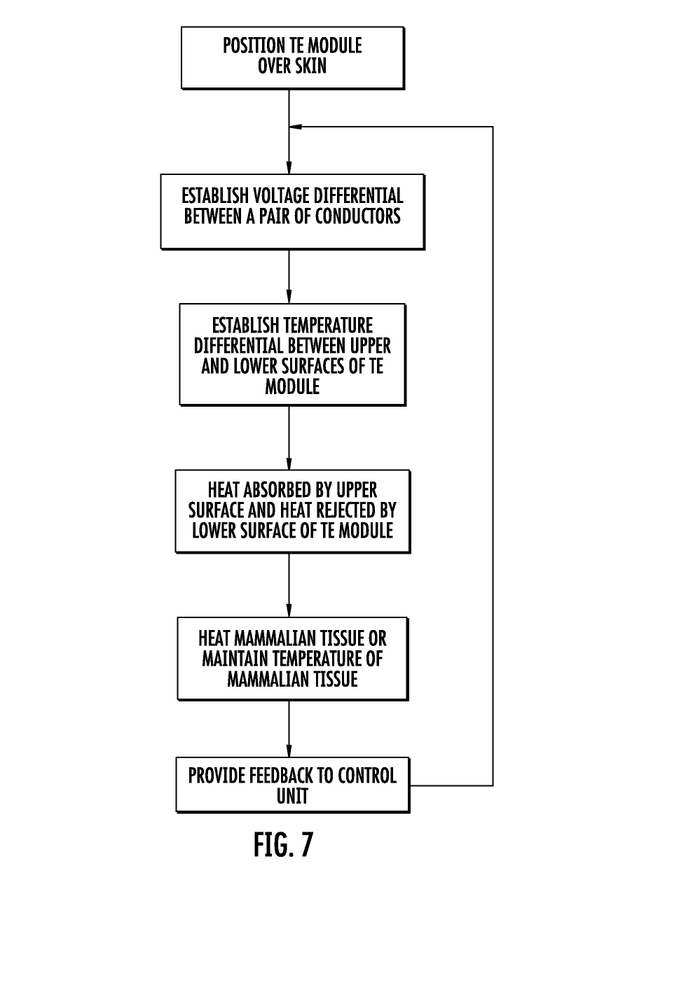

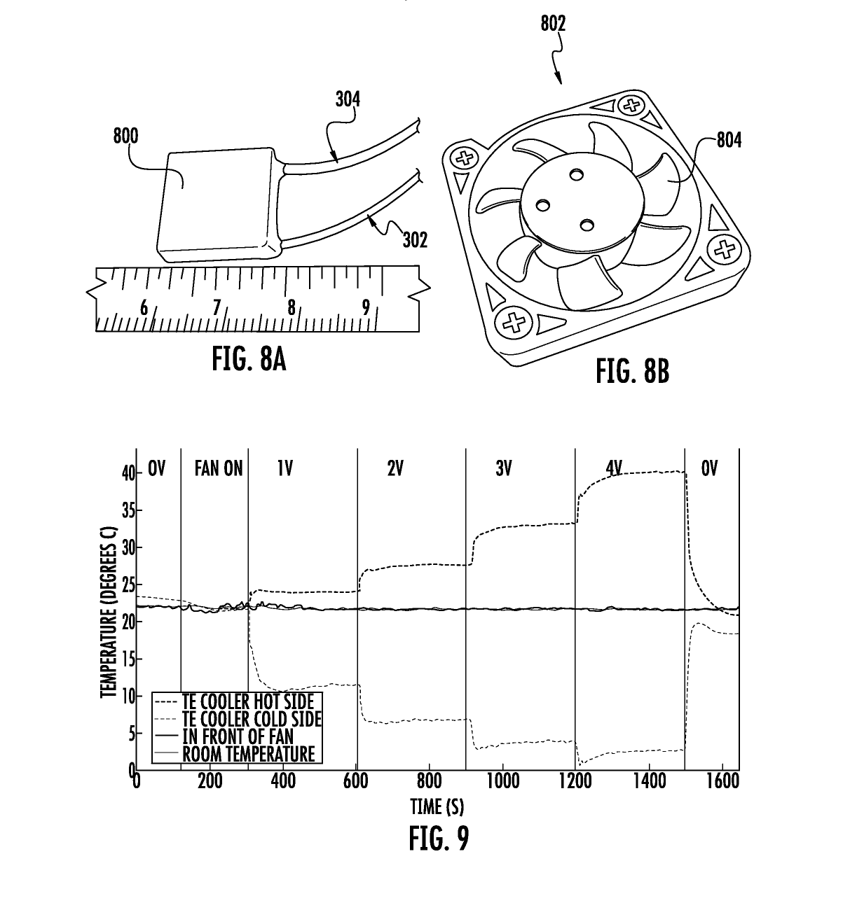

Referring to FIG. 8A, an example bismuth thermoelectric cooler (TEC) is shown. The TEC 800 optionally operates at an internal electrical resistance in the range of about 4 to 6 ohm. Referring to FIG. 8B, an example finned heat sink is shown. The finned sink 802 is optionally applied to the upper surface 316 of the TEC 800. The finned sink 802 may provide a cooling fan 804 to optionally provide forced convection heat transfer on the upper surface 316 of the TEC 800. Optionally, the TEC 800 and finned sink 802 may be attached to a substrate and may be arranged in a variety of spatial orientations. The electrical resistance of the TEC 800 may optimally be in the range of 4 to 6 Ohms in order to balance the amount of current that can be generated by an applied source voltage to cause heat flow from the cooler side to the warmer side and of the rate of internal energy generation within the device by ohmic heating. Both heat flows may optionally be dissipated from the warmer side of the TEC 800. Therefore, it may be desirable to remove heat from the warmer side surface to environmental room temperature air. One of the preferred embodiments may be to remove heat via forced convection heat transfer in combination with a finned heat sink applied to the warmer surface. Forced convection is caused by the flow of air motivated by a fan directed toward the warmer surface and heat sink. Optionally, each TEC 800 may have a dedicated fan, or a single fan may supply a flow of forced convection air for multiple TEC 800 modules.

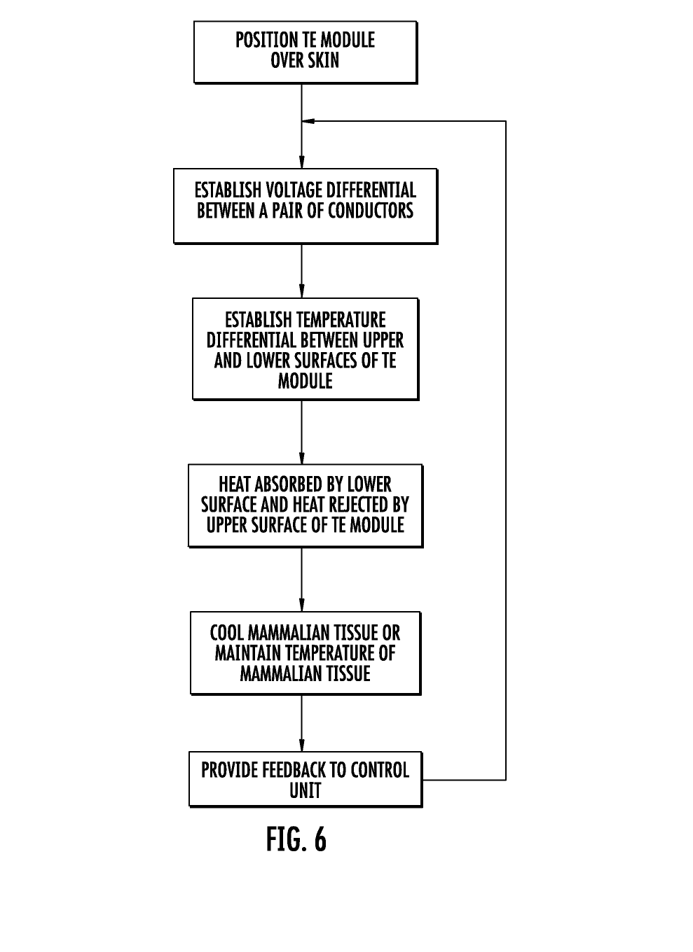

Referring now to FIGS. 6 and 7, flowcharts of applying cooling and heating effects with example TE modules are shown. In FIG. 6, the steps for providing a cooling effect with an example TE module 104 are shown. After positioning an example TE module 104 over the skin, a voltage differential may be established between a pair of conductors (304 and 302). The voltage differential may establish a temperature differential based on the direction of the applied voltage. For example, the electrical line 302 may provide a positive voltage and the electrical line 304 may provide a negative voltage. Subsequently, the temperature differential may be such that heat may be rejected by the upper surface and heat may be absorbed by the lower surface. The mammalian tissue or skin upon which the TE module 104 is positioned may optionally be cooled or the temperature maintained. Any feedback from the skin or lower surface, or proximate to either, such as temperature information, may be communicated to the control unit 401 and may be used to adjust or maintain application of heat or cooling by the TE module.

In FIG. 7, the steps for providing a heating effect with a TE module 104 are shown. After positioning an example TE module 104 over the skin, a voltage differential may be established between a pair of conductors (304 and 302). The voltage differential may establish a temperature differential based on the direction of the applied voltage. For example, the electrical line 304 may provide a positive voltage and the electrical line 302 may provide a negative voltage. Subsequently, the temperature differential may be such that heat may be absorbed by the upper surface and heat may be rejected by the lower surface. The mammalian tissue or skin upon which the TE module 104 is positioned may optionally be heated or the temperature maintained. Any feedback from the skin or lower surface, or proximate to either, such as temperature information, may be communicated to the control unit 401 and may be used to adjust or maintain application of heat or cooling by the TE module.

The cooling and heating temperature and power of the TE module 104 may be controlled by adjustment of the magnitude and sign of the applied voltage. In other words, current flows within the thermoelectric module 104, and one side of the TE module 104 is cooled and the other heated. Where the current flow is reversed, the hot and cold sides reverse as well. For example, the heat may be rejected on the side of the TE module 104 that is hot, and the heat may be absorbed on the side of the TE module 104 that is cold. The sensor unit 308 optionally may provide feedback such as, for example, the temperature at the control surface or the temperature on the lower surface 318 of the TE module 104. The sensor unit 308 may be connected or communicable with a control unit 401 and may provide feedback to the control unit.

Various entities design, market, and manufacture thermoelectric devices that can be used in the described modules. For example, these entities include but are not limited to Gentherm (Northville, Mich.), BSST, LLC (Northville, Mich.), Ferrotec (Bedford, N.H.), and Crystal Ltd. (Moscow, Russia), among others.

In general, and not meant to be limiting, an example TE module 104 may include thermoelectric couples, which further include n-type thermoelectric elements, which contain free electrons, and p-type thermoelectric elements, which contain free holes. These n-type and p-type thermoelectric elements are wired electrically in series and thermally in parallel. The n-type and p-type thermoelectric elements may include doped semiconductor elements, such as, for example, suitably doped bismuth telluride. The n-type and p-type segments may be connected by shunts to form an electric circuit. Electric circuit is propagated by electrons in n-type materials and by holes (traveling in the opposite direction) in p-type materials. P-type materials may further include semiconductors, metals, or semimetals.

For example, the TE module 104 may include electrical lines 302 and 304. The electrical lines 302 and 304 may introduce an input voltage along electrical line 302 and an output voltage along electrical line 304. The voltages along electrical lines 302 and 304 may be applied in a lateral direction such that a temperature change 310 may result in the vertical direction. Where voltage is applied, for example, in a direction across the p-n junction, electron/hole pairs are created in the vicinity of the junction. Electrons may flow away from the junction in the n-type material, and holes flow away from the junction in the p-type material. The energy to form them comes from the junction region, cooling it. On the opposite end, electrons and holes stream toward junctions where pairs recombine. In other words, all junctions on one side of the TE module 104 may heat, while all of the junctions on the other side of the TE module may cool.

The various operating parameters of the TE module 104 may be adapted or configured to various clinical settings or other operating circumstances. Furthermore, the operating parameters may differ depending on whether one or more TE module 104 is being operated on a cooling or heating setting.

For example, the operating parameters for the cooling setting may include operating at temperatures between about 5° C. and about 30° C. For the cooling setting, the heat flux may operate at about 1 kW/m². For the heating setting, the optimal operating temperature may include a maximum temperature of about 43° C. This maximum optimal operating temperature may avoid burning of the subject's skin. The optimal operating temperature may be greater than about 43° C., wherein thermally insulating protection of some type is provided for protection of the skin from burning or burns. The convection coefficient for the heating setting for the heat rejected 312 may include at least about 35 Watts/m²·K.

The figure of merit (zT), for example, provides the maximum efficiency of a thermoelectric material. The figure of merit may be represented as follows:

$$zT = \frac{{α}^{2} T}{ρκ}$$

zT depends on α (Seebeck voltage), absolute temperature (T), electrical resistivity (ρ), and thermal conductivity (κ). The figure of merit (zT) for the thermoelectric material provided in the present disclosure may include about 1.0, and the sink temperature may include about 300 K.

Each TE module 104 may vary temperature between the heating and cooling settings and in any temperature therebetween. Furthermore, each TE module 104 may vary temperature between a cooling setting and a heating setting and any temperature therebetween over a period of time. The temperature differential in a TE module 104 may be altered based on a change in the magnitude and direction of the applied voltage. Where the direction of the applied voltage is changed, the temperature of the lower and upper portions of the TE module 104 may also be changed.

The example devices optionally further include a connecting substrate 102 and at least two thermoelectric modules 104 are fixed to the substrate 102. An example substrate and example thermoelectric modules fixed thereto is shown in FIG. 1. The fixation to the substrate 102 orients the position of at least two thermoelectric modules 104 relative to each other. Optionally, the connecting substrate 102 is flexible. Optionally, at least a portion of the flexible substrate 102 and at least two thermoelectric modules 104 are conformable to a portion of the mammal anatomy or tissue. Optionally, the orientation of one or more thermoelectric modules 104 is adjustable in three dimensions. Optionally, each of the thermoelectric modules 104 are fixed to the substrate 102 to orient their position relative to each other.

Referring again to FIG. 1, an example array 106 of thermoelectric modules 104 are shown arranged on a flexible substrate and placed on a subject's knee. The predetermined spatial orientation 106 may include two or more TE modules 104. The TE modules 104 may optionally be arranged on a flexible substrate 102. The size of the TE modules 104 may be adapted to conform to or meet the needs of the situation or device, as appropriate. Thus, the array 106 may include one TE module 104 or as many TE modules 104 as appropriate, depending on the size of the tiles and the size of desired coverage, for example.

Optionally, the connecting substrate 102 is a mesh. In examples where the connecting substrate 102 is flexible, it may include a mesh material, a metallic material, or other appropriate material. An example embodiment of the flexible substrate 102 may include a thin, open-celled foam impregnated with a hydrogel having shear thinning properties. The shear thinning properties may allow the hydrogel to be introduced into the cells of the foam while in a state of low viscosity and subsequently to be retained within the foam when in a state of high viscosity. The shear thinning properties may allow the thermal conductivity enhancing material to be introduced into the matrix of the foam in a state of low flow resistance and then to remain in a state of high flow resistance to provide an overall increase in the heat transfer capacity of the foam. Further, the mesh material may optionally include a polymer to enhance the capacity to transmit heat across a temperature gradient. The flexible substrate 102 may allow the array 106 of TE modules 104 to adapt or conform to the geometric contour of the area of the subject's body to which the flexible substrate 102 and at least two TE modules 104 may be applied.

The TE modules 104 may optionally be attached to the flexible substrate 102 with an adhesive or other appropriate fastening or attaching means. The properties of the flexible substrate 102, such as, for example, the optical properties, the thermal properties, the size of the substrate, and the thickness of substrate 102, may optionally be configured and tailored for appropriate circumstances. The various properties of the flexible substrate 102 optionally present as little resistance as possible to the flow of heat and to flexure to conform to the geometry of a three dimensional surface to which the flexible substrate 102 is applied. Optionally, optical transparency of the flexible substrate 102 may be appropriate to facilitate visual inspection of the tissues in open areas between TE modules 102.

Optionally, the connecting substrate 102 is thermally conductive. The flexible substrate 102 may optionally include a material for lateral thermal conductivity to allow for transfer of heat to the skin of the subject. Examples of such material include wire or mesh. The wire or mesh optionally have a relatively large intrinsic thermal conductivity integrated into the flexible substrate 102 to facilitate laterally spreading of heat and minimizing of lateral temperature gradients. Such material may include a metal, a polymer, or any other highly conductive material. Optionally, the connecting substrate 102 has a low force deformability.

Activation of the thermoelectric modules 104 can be varied in the temporal and spatial dimensions. For example, at least two of the thermoelectric modules 104 are selectively operable in a temporal dimension. In another example, each of the modules 104 is selectively operable in a temporal dimension. At least two of the thermoelectric modules 104 can also be selectively operable in a spatial dimension and these are also optionally selectively operable in the temporal dimension. In some examples, each of the modules is selectively operable in a spatial dimension.

One example of a selective temporal and spatial orientation may include applications of TE modules 104 for cryotherapy. In a situation involving cryotherapy, there may be a therapeutic objective of providing across a treatment area or treatment site, an overall reduction of temperature for pain analgesia reduction in intensity of the inflammatory process and an overall reduction of blood flow to control swelling. However, reduced blood flow may cause ischemia that, where sustained for an extended period of time, may lead to ischemic injury, reperfusion injury, and reduced healing rates from the lack of oxygen and nutrients and a buildup of metabolic byproducts. Providing a short burst of fresh blood perfusion can prevent the occurrence of ischemia-derived injuries. Localized rewarming of cooled tissue may reestablish a local high rate of blood perfusion, obviating ischemia and its injury cascade. Localized rewarming of cooled tissue may also elevate the dissociation of oxygen molecules from hemoglobin when they are delivered to the blood capillaries so that a state of cold induced hypoxia may be overcome, thereby enhancing the healing of injured tissues in the treatment area. Following a brief episode of rewarming, the tissue temperature may return to low values with their therapeutic advantages.

The ability of thermoelectric modules to alternatively be cooled and warmed cyclically may provide advantages for safer therapy of tissues than can be achieved by continuous cooling. Two or more thermoelectric modules 104 may be programmed to execute a spatial and temporal pattern of cyclic cooling and warming so that the overall effect on a total treatment area is therapeutic cooling, in combination with blocking the persistence of an ischemic state or a hypoxic state that may lead to tissue injury.

Another example of selective temporal and spatial operation occurs in applications for remediation of the effects of menopause on thermoregulation that are manifested symptomatically as hot flashes. In this case, the thermoelectric modules 104 may produce a control rather than an energetic input to mammalian tissue. A single or plurality of thermoelectric modules may be placed on or proximate to the skin to be activated for cooling during an anticipated or actual hot flash, thereby generating a signal to offset the vasodilation and sweating action of the thermoregulatory system. Two or more TE modules 104 may be programmed to operate uniformly or to cycle temporally and/or spatially to provide a maximum effect to obviate the hot flash symptoms. The thermoelectric modules may be positioned optimally to thermally contact peripheral thermoregulatory control tissue on the body surface to exert the desired thermoregulatory control input to offset the vasodilation and sweating actions.

In the example devices, at least two of the thermoelectric modules 104 are positionable in overlying registration with the mammalian tissue. Optionally, the mammalian tissue is skin.

The devices optionally further include a barrier substrate positioned between the mammalian tissue and the thermoelectric modules 104. The barrier substrate is optionally disposable. For example, the barrier substrate may attach to the flexible substrate 102. The barrier substrate may optionally be configured such that the properties of the barrier substrate may be tailored or configured for certain or appropriate circumstances. In the clinical setting, for example, once the barrier substrate, flexible substrate 102, and TE modules 104 have been used, the barrier substrate may be disposed thereof by appropriate means. The flexible substrate 102 and TE modules 104 may then be used again for another procedure or otherwise.

The example devices include thermoelectric modules 104 that have a length dimension, a width dimension and a thickness dimension. The length dimension is optionally equal to the width dimension. The length dimension is optionally about 2.0 centimeters. In other examples, the length dimension is not equal to the width dimension. The dimensions of the TE modules 104 may be varied and adapted as appropriate. Optionally, the size of the flexible substrate 102 may be adapted or varied as appropriate.

For example, in a situation where the surface to apply a heating or cooling effect with a flexible substrate 102 and at least two TE modules 104 is large with a diverse contour, the flexible substrate 102 may be large and the size of the TE modules 104 may optionally be small to adapt to the diverse contour of the body region. In a situation where the surface to apply a heating or cooling effect with a flexible substrate 102 and at least two TE modules 104 is large with a non-diverse contour, the flexible substrate 102 may be large and the size of the TE modules 104 may optionally be large to adapt to the non-diverse contour of the body region. In a situation where the surface to apply a heating or cooling effect with a flexible substrate 102 and at least two TE modules 104 is small with a diverse contour, the flexible substrate 102 may be small and the size of the TE modules 104 may optionally be small to adapt to the diverse contour of the body region. In a situation where the surface to apply a heating or cooling effect with a flexible substrate 102 with at least two TE modules 104 is large with a non-diverse contour, the flexible substrate 102 may be small and the size of the TE modules 104 may optionally be large to adapt to the non-diverse contour of the body region.

For example, larger TE modules 104 attached to a flexible substrate 102 may adapt more easily to areas of the subject's body in which the geometric contour is not as diverse. In the alternative, smaller TE modules 104 may adapt more easily to areas of the subject's body in which the geometric contour is more diverse, such as, for example, the knee region.

In the example devices, each thermoelectric module 104 is optionally spaced from each other thermoelectric module 104, and optionally, the spacing between the thermoelectric modules 104 is the same. The spacing between the adjacent TE modules 104 may be such that the flexible substrate 102 may deform to adapt to a body contour without causing the adjacent TE modules 104 to come into physical contact with each other.

The described devices optionally further include a plurality of sensor units 308, wherein each sensor unit 308 senses temperature proximate to at least one thermoelectric module 104. An example TE module 104 with an example sensor unit 308 is shown in FIG. 3. Optionally, each thermoelectric module 104 has an associated sensor unit 308. Optionally, the devices include a control unit 401, having at least one processing device and a plurality of sensor units 308. Each thermoelectric module 104 is associated with at least one of the sensor units 308 and the sensor units 308 sense temperature and communicate the sensed temperature to the control unit 401 for processing. The sensor units 308 are optionally positioned at the lower surface 318 of one or more thermoelectric modules 104 when the lower surface 318 is more proximate the tissue than the upper surface 316. The sensor units 308 optionally sense the temperature of the tissue or of the lower surface 318. The sensed temperature is optionally communicated to the control unit 401 where it is used to adjust the operational characteristics of one or more thermoelectric modules 104.

The control unit 401 may include standard embedded microcontrollers. The control unit 401 may also include a computer or other processing system. The control unit 401 may allow for each TE module 104 to be controlled independently and operated independently. Referring again to FIG. 4, an example control system 400 is shown. The example control system 400 may include a control unit 401. The processing system may also process temperatures communicated by sensor units 308, as well as the temperature of the subject's skin 406 and the core temperature of the subject 404.

Based on the clinical setting, individual preferences, or therapeutic goals, the temperature reading or other feedback from the sensor unit 308 may allow adjustment of various operating parameters for one, some, or all of the TE modules 104 in a particular embodiment.

The control unit 401 may also include one or more apparatuses for monitoring the temperature of the skin 406 of the subject or patient. These one or more apparatuses for monitoring the temperature of the skin 406 of the subject or patient may be placed at various parts of the subject's body, such that multiple temperatures may be obtained and provided as feedback to the control unit 401. The control unit 401 may also include an apparatus for monitoring the core temperature 404 of the subject.

The control unit 401 may be controlled automatically via a processing system or manually by inputs from a user. Depending on the specific clinical and/or therapeutic needs, the temperature of the one or more TE modules 104 may be individually altered or changed. For example, in an example spatial orientation of at least two TE modules 104, each TE module may include a different heating or cooling setting.

Thus the methods, devices and systems described herein can optionally be implemented via a processing system such as a general-purpose computing device in the form of a computer. The components of the computer can include, but are not limited to, one or more processors or processing units, a system memory, and a system bus that couples various system components including the processor to the system memory.

The system bus may represent one or more of several possible types of bus structures, including a memory bus or memory controller, a peripheral bus, an accelerated graphics port, and a processor or local bus using any of a variety of bus architectures. By way of example, such architectures can include an Industry Standard Architecture (ISA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. The bus, and all buses specified in this description, can also be implemented over a wired or wireless network connection and each of the subsystems, including the processor, a mass storage device, an operating system, application software, data, a network adapter, system memory, an Input/Output Interface, a display adapter, a display device, and a human machine interface, can be contained within one or more remote computing devices at physically separate locations, connected through buses of this form, in effect implementing a fully distributed system.

The computer typically includes a variety of computer readable media. Such media can be any available media that is accessible by the computer and includes both volatile and non-volatile media, removable and non-removable media. The system memory includes computer readable media in the form of volatile memory, such as random access memory (RAM), and/or non-volatile memory, such as read only memory (ROM). The system memory typically contains data such as data and and/or program modules such as operating system and application software that are immediately accessible to and/or are presently operated on by the processing unit. The computer may also include other removable/non-removable, volatile/non-volatile computer storage media. A mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules can be stored on the mass storage device, including by way of example, an operating system and application software. Each of the operating system and application software (or some combination thereof) may include elements of the programming and the application software. Data can also be stored on the mass storage device. Data can be stored in any of one or more databases known in the art. Examples of such databases include, DB2®, Microsoft® Access, Microsoft® SQL Server, Oracle®, mySQL, PostgreSQL, and the like. The databases can be centralized or distributed across multiple systems. Application software may include instructions for determining and communicating the position of the model fetus in the system and for advancing the model fetus in the system.

A user can enter commands and information into the computer via an input device. Examples of such input devices include, but are not limited to, a keyboard, pointing device (e.g., a “mouse”), a microphone, a joystick, a serial port, a scanner, and the like. These and other input devices can be connected to the processing unit via a human machine interface that is coupled to the system bus, but may be connected by other interface and bus structures, such as a parallel port, game port, or a universal serial bus (USB).

The computer can operate in a networked environment using logical connections to one or more remote computing devices. By way of example, a remote computing device can be a personal computer, portable computer, a server, a router, a network computer, a peer device or other common network node, and so on. Logical connections between the computer and a remote computing device can be made via a local area network (LAN) and a general wide area network (WAN). Such network connections can be through a network adapter. A network adapter can be implemented in both wired and wireless environments. Such networking environments are commonplace in offices, enterprise-wide computer networks, intranets, and the Internet.

An implementation of application software may be stored on or transmitted across some form of computer readable media. Computer readable media can be any available media that can be accessed by a computer. By way of example, and not limitation, computer readable media may comprise “computer storage media” and “communications media.” “Computer storage media” include volatile and non-volatile, removable and non-removable media implemented in any method or technology for storage of information such as computer readable instructions, data structures, program modules, or other data.

Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer. An implementation of the disclosed method may be stored on or transmitted across some form of computer readable media.

The processing of the disclosed methods can be performed by software components. The disclosed methods may be described in the general context of computer-executable instructions, such as program modules, being executed by one or more computers or other devices. Generally, program modules include computer code, routines, programs, objects, components, data structures, etc. that perform particular tasks or implement particular abstract data types. The disclosed method may also be practiced in grid-based and distributed computing environments where tasks are performed by remote processing devices that are linked through a communications network. In a distributed computing environment, program modules may be located in both local and remote computer storage media including memory storage devices.

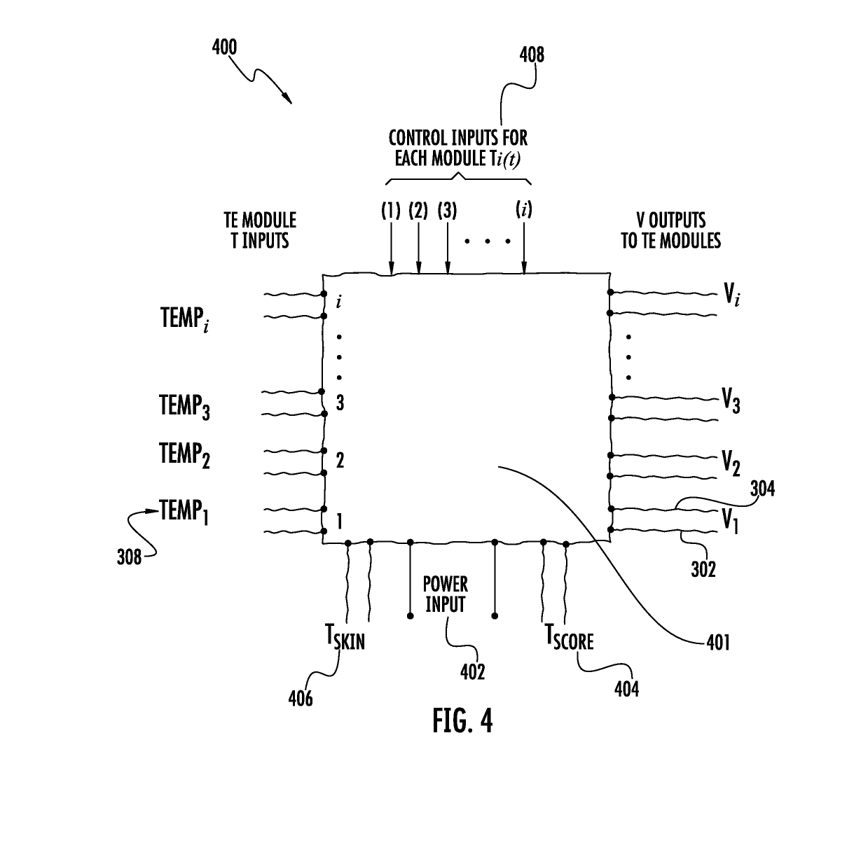

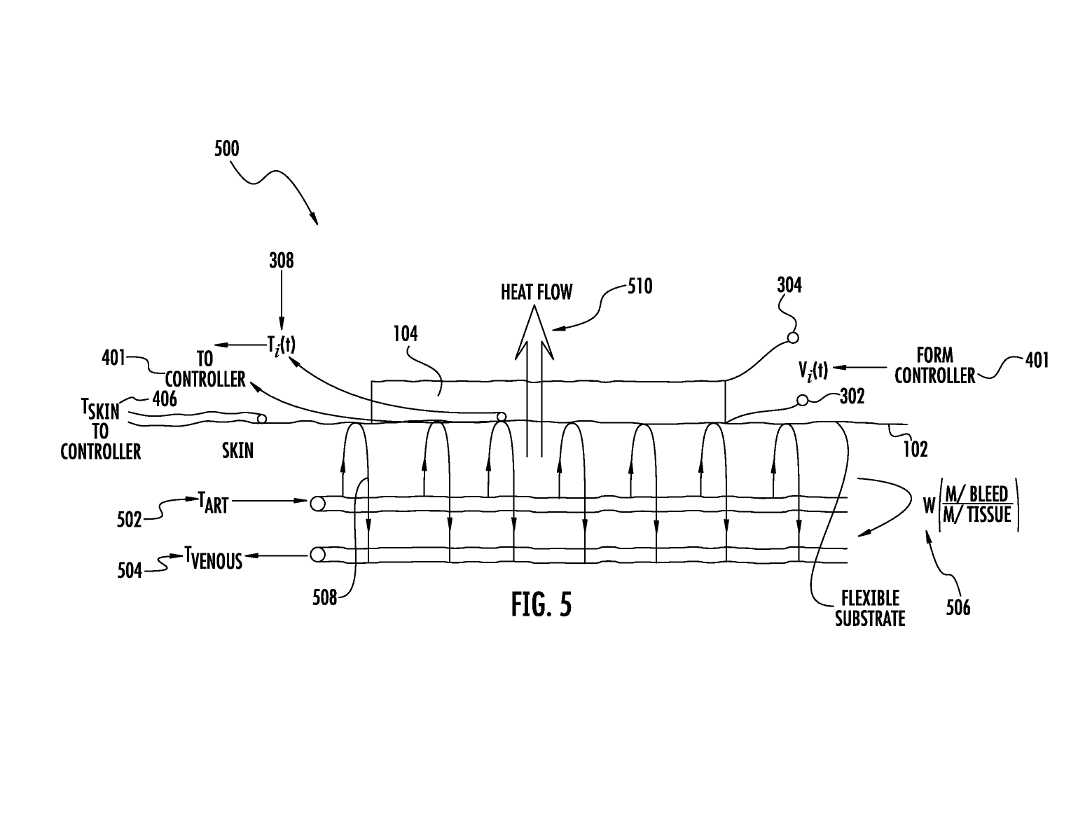

Referring to FIG. 5, an example TE module situated on the skin of a subject is shown. The example TE module 104 may be attached to a flexible substrate 102. The sensor unit 308 may optionally be connected or communicable with the control unit 401. As shown in FIG. 5, the initial temperature of the arterial blood vessels 502 can include an initial temperature. The heat flow 510 may cause the flow of blood from the arterial blood vessels to the venous blood vessels 508. The blood flow 506 (ω) may be denoted as such:

$$ω ( \frac{{mL}_{blood}}{{mL}_{tissue} \cdot sec} )$$

The flow of blood may also be expressed as Volume per second. The temperature of the venous blood vessels 504 may include a temperature that is different than the initial temperature of the arterial blood vessels 502.

Also provided herein are temperature sources to drive heat transfer processes, wherein the temperature source is a thermoelectric (TE) material. For example, the sources are used to drive heat transfer processes in mammalian tissue. Optionally, the TE material is positioned at the site of treatment on a mammal. Optionally, the TE material is in thermal communication with mammal tissue without need for an intermediate convective transport medium such as flowing water. Optionally, the TE material is configured into a plurality of TE tiles 104 each of which is individually and independently controllable.

As described throughout, individual TE tiles 104 are optionally mountable onto a flexible membrane 102 that allows the aggregate TE tiles 104 to conform to the shape of a mammalian surface to which they are applied. The flexible membrane 102 optionally allows for differences in three-dimensional orientation between adjacent TE tiles 104. The flexible membrane 102 optionally has properties of high thermal conductivity to provide thermal communication between a TE material and a mammalian tissue surface. Optionally, the flexible membrane 102 has a low force elastic deformability to conform to the geometry of a mammalian surface to increase the contact area between the TE material and the mammalian surface. The flexible membrane 102 is optionally tailored by addition of chemical constituents, tailoring of stoichiometry, or method of curing such that heat transfer, or other properties such as optical or electrical, may be varied.

Also provided is a flexible elastically deformable membrane 102, wherein the membrane increases the surface contact area between a curved mammalian surface and a contacting rigid TE material. The flexible membrane 102 optionally has an open pore structure that can be loaded with a shear thinning gel. Optionally, the membrane 102 has an open pore structure that can be loaded, injected, or impregnated with a fluid/material that affects the thermal, optical, electrical, or other properties in the manner of the membrane. Optionally the material is copper slurry or ultrasound gel.

Also provided is a control system 401 for TE material tiles 102 that enables temporal and spatial modulation of the temperature applied to the mammalian surface with feedback from individual thermal sensors 308 associated with each tile. Optionally, all of the TE tiles 104 are programmed to produce a positive heat flux. Optionally, all of the TE tiles 104 are programmed to produce a negative heat flux. Optionally, the TE tiles 104 are programmed to provide both positive and negative heat fluxes across the tiles in a checkerboard or similar fashion to illicit responses from the body's thermal receptors and therefore impact neural control and sensation of the affected tissues. Optionally, the TE tiles 104 are controlled by individual PID feedback calculations based on sensors 308 on the surface of the mammalian tissue and/or in the intervening material layers of the device. Optionally, the TE tiles 104 are controlled by individual control feedback calculations based on temperature sensors at the surface of the mammalian tissue and/or in the intervening material layers of the device. Optionally, the TE tiles 104 are controlled by individual control calculations based on heat flux sensors at the surface of the mammalian tissue and/or in the intervening material layers of the device. Optionally, the TE tiles 104 are controlled by individual control calculations based on blood perfusion sensors at the surface of the mammalian tissue.

Also provided is a programmable control component that can provide and enforce a pattern of temperature modulation in time and position on the surface of a mammalian tissue. Also provided is a method of powering of a bioheat transfer device with only an electrical source. Also provided is a method of cooling the outer surface of a bioheat transfer device with a fan. Also provided is a method of cooling the outer surface of a bioheat transfer device with heat sink topologies, such as fins. Also provided is a method of cooling the outer surface of a bioheat transfer device with both heat sink topologies, such as fins, and fans. Also provided is a bioheat transfer device that consists of only a single physical unit. Also provided, is a bioheat transfer device that has no liquid flow tubes. Also provided is a switch to operate a TE material only when subject tissue is to be cooled or heated. Also provides is a TE material for application to a surface of mammalian tissue to cause a thermal load for substantially altering the temperature and energy stored in a tissue volume.

Further provided is a TE device consisting of a plurality of TE tiles 104, wherein a tile 104 is a single rigid, planar, and independently operable TE device. Optionally, the TE tiles 104 are on the order of 1 cm² or 2 cm² or 3 cm². Optionally the device includes a plurality of independent TE tiles 104 arranged in a parallel array. Optionally, the array exists as a rigid plane. Optionally, the array is mounted on a flexible membrane 102. Optionally, the individual TE tiles 104 are arranged with significant space between each other, but are attached to the flexible membrane 102 in a manner so as they are freely moveable with respect to each other as the membrane 102 moves. Optionally, the free movement is intended to surround a curved or highly irregular mammalian surface and produce a substantial degree of contact. Optionally, the individual TE tiles 104 are arranged with simple hinge structures between adjacent tiles 104 in addition to being attached to the flexible membrane 102 in a manner so as they may move with respect to each other as the membrane 102 moves. Optionally, the movement surrounds a curved or highly irregular mammalian surface and produces a substantial degree of contact while maintaining structural integrity.

The described TE devices 104 can heat or cool dynamically with spatio-temporal resolution. Optionally, the individual TE tiles 104 are bipolar or have a thermal polarity different by row, column, or in some pattern. Alternating cold/hot tiles 104 optionally generates a unique or high-magnitude physiological response that may be altered over the course of a treatment according to a protocol designed to achieve a therapeutic outcome.

In the described TE devices the flexible membrane 102 optionally includes memory foam. Optionally, the flexible membrane 102 provides a form factor and methods to modulate thermal properties of the device. This modulation is optionally achieved by altering membrane 102 thickness. This modulation is optionally achieved via impregnation with gels of defined thermal, optical, or electrical properties. This modulation is optionally achieved by altering the porosity, density, specific heat, insulation, and heat diffusion anisotropy.

In the described devices the TE component is optionally reusable and an intervening flexible membrane 102 is optionally disposable.

Also provided are methods of affecting temperature of mammalian tissue. The methods optionally include providing a device as described herein and positioning the device such that at least two of the thermoelectric modules 104 are in overlying registration with the tissue with the lower surface 318 proximate the tissue relative to the upper surface 316. An electric current flow is initiated through at least one of the thermoelectric modules 104 to cause absorption of heat by the lower surface 318 of the thermoelectric module 104 from the mammalian tissue and rejection of heat by the upper surface 316 of the thermoelectric module 104 to cool the mammalian tissue.

The methods optionally further include initiating an electric current flow through at least one additional of the thermoelectric modules 104 positioned in overlying registration with the tissue to cause absorption of heat by the lower surface of the thermoelectric module 104 from the mammalian tissue and rejection of heat by the upper surface 316 of the thermoelectric module to cool the mammalian tissue.

The methods optionally further include initiating an electric current flow through at least one additional of the thermoelectric modules 104 positioned in overlying registration with the tissue to cause rejection of heat by the lower surface 318 of the thermoelectric module 104 into the mammalian tissue and absorption of heat by the upper surface 316 of the thermoelectric module 104 to heat the mammalian tissue.

Also provided are methods of affecting temperature of mammalian tissue that include providing a device as described herein and positioning the device such that at least two of the thermoelectric modules 104 are in overlying registration with the tissue with the lower surface 318 proximate the tissue relative to the upper surface 316. An electric current flow is initiated through at least one of the thermoelectric modules 104 to cause rejection of heat by the lower surface 318 of the thermoelectric module 104 from the mammalian tissue and absorption of heat by the upper surface 316 of the thermoelectric module 104 to heat the mammalian tissue.

The methods optionally further include initiating an electric current flow through at least one additional of the thermoelectric modules 104 positioned in overlying registration with the tissue to cause rejection of heat by the lower surface 318 of the thermoelectric module 104 from the mammalian tissue and absorption of heat by the upper surface 316 of the thermoelectric module 104 to heat the mammalian tissue.

The methods optionally further include initiating an electric current flow through at least one additional of the thermoelectric modules 104 positioned in overlying registration with the tissue to cause absorption of heat by the lower surface 318 of the thermoelectric module 104 into the mammalian tissue and rejection of heat by the upper surface 316 of the thermoelectric module 104 to cool the mammalian tissue.

The methods described herein optionally include activating one or more thermoelectric module 104 is selectively by initiating the current through that module at a predetermined time. The predetermined time for activation is optionally different for activation of one or more modules 104. Optionally, the predetermined time of activation is based on feedback from mammalian tissue or from the mammal.

### EXAMPLES

### Example 1

### Incremental Applied Voltage

The thermoelectric modules were used at incremental applied voltages. Both the upper and lower surfaces of the thermoelectric module were exposed to air at room temperature. The upper surface was also exposed to forced convection of air over a finned heat sink. Referring to FIG. 9, the test results are shown. As shown, the lower surface of the thermoelectric module remained below 20° C., and the upper surface did not reach the temperature threshold of 43° C., which is the temperature at which thermal injury may occur should the thermoelectric module be contacted by a person.

### Example 2

### Module Applied to Skin Surrogate Material

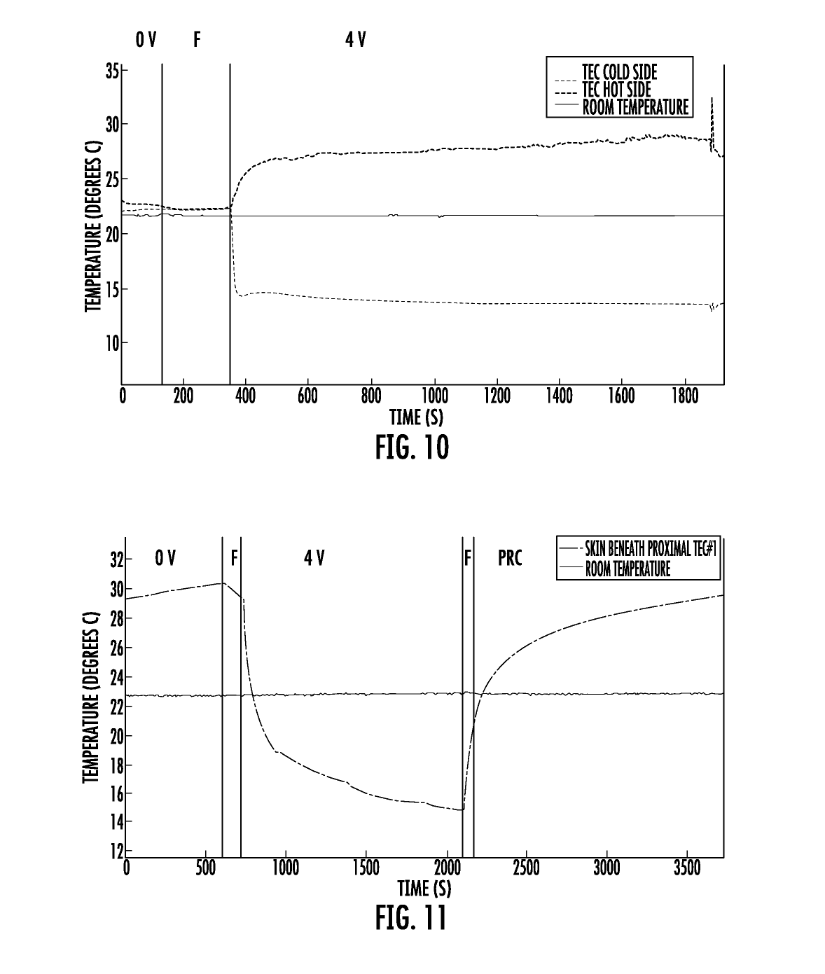

Thermoelectric modules were applied to a skin surrogate material with an initial temperature of 22° C. The skin surrogate material used has properties similar to those of human tissue, but without the metabolism and blood flow. Referring to FIG. 10, the test results are shown. The operating cool temperature on the lower surface was achieved in less than one minute, and the temperature on the upper surface of the thermoelectric module remained below 43° C.

### Example 3

### Module at Incremental Voltages Applied to Human Skin

Thermoelectric modules were used at an applied voltage of 4V with forced convection to room temperature air to cool human skin. Referring to FIG. 11, the results are shown. As shown, the operating temperature achieved on the surface of the skin is below 16° C., which is below the preferred therapeutic range for both lowering core temperature by cooling of blood flowing through glabrous skin and for applying cryotherapy. The applied voltage of 4V is a preferred applied voltage for the thermoelectric module demonstrated in these results, having an operating resistance of approximately 4 Ohms. Heat removal from the warmer side of the thermoelectric module was achieved via forced convection air flow over a finned heat sink with a dedicated fan.

### Example 4

### Module Applied to Skin Surrogate Material

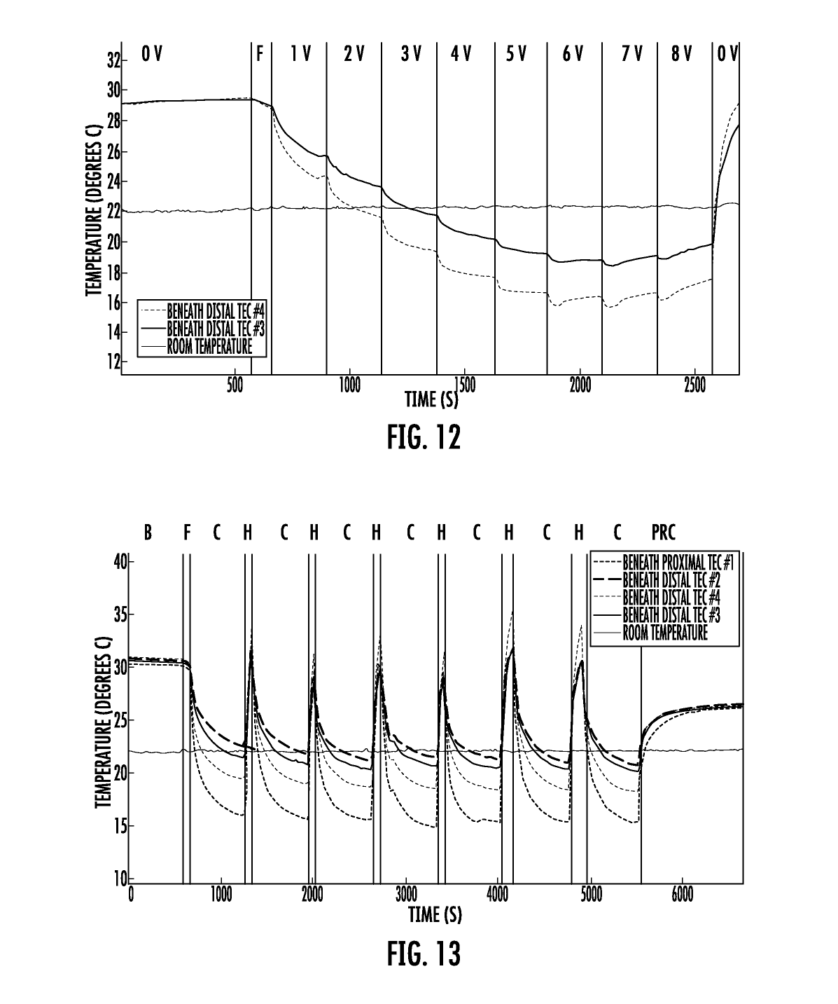

Thermoelectric modules were used at incremental applied voltages to cool human skin. The range of voltages included up to 8V. Referring to FIG. 12, the results are shown. This data shows that there is a preferred operating voltage for a given thermoelectric module having a given internal electrical resistance. As higher voltages are applied, the internal ohmic heating of the thermoelectric module increases according to the relationship of P=i²R. Up to a certain level, increasing current causes a larger temperature difference to be created across the thermoelectric module. However, because the internal power dissipation increases according to the square of the current, at levels of current above the preferred level, the temperature difference created by the Peltier effect is overcome by the internal electrical dissipation. Therefore, for a specific module design, the preferred operating state can be determined.

### Example 5

### Module Applied to Skin with Heating and Cooling Cycles

Thermoelectric modules were used with heating and cooling cycles for thermoelectric modules mounted on human subject skin. The results are shown in FIG. 13. As shown in FIG. 13, there were several phases involved, as follows: B, for baseline data acquisition; F, for application of only the forced convection cooling fan to the upper surface of the thermoelectric device; C, for the voltage polarity to cause cooling of the skin surface; and H, for a voltage polarity to cause heating on the skin surface. As further shown, the thermoelectric module was capable of operating between about 16° C. and 33° C. during the cooling and heating cycles. Preferred lowest operating temperatures for cryotherapy may be between 15° C. and 25° C. applied to nonglabrous skin and between 20° C. and 25° C. applied to glabrous skin and for core temperature reduction. For both cryotherapy warming and core temperature warming, the operating temperature is preferably between the nominal skin temperature and 43° C., which is the threshold for thermal injury causation.

Many modifications and other embodiments of the disclosure set forth herein will come to mind to one skilled in the art to which this disclosure pertains having the benefit of the teachings presented in the foregoing description. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1-74. (canceled)

75. A device for heating, cooling, or maintaining temperature of mammalian tissue, the device comprising:
a. at least one thermoelectric module, the module having an upper and a lower surface, wherein the module is selectively operable to reject heat from one of the upper or lower surfaces and to absorb heat from one of the upper or lower surfaces;
b. wherein the lower surface is positionable in proximity to and overlying mammalian tissue relative to the upper surface such that the lower surface absorbs heat from the proximate, underlying mammalian tissue, or such that the lower surface rejects heat into the proximate, underlying mammalian tissue.

76. The device of claim 75, further comprising a plurality of thermoelectric modules, wherein each thermoelectric module has an upper and a lower surface, wherein each module is selectively operable to reject heat from one of its upper or lower surfaces and to absorb heat from one of its upper or lower surfaces and wherein the lower surface of each module is positionable in proximity to and overlying mammalian tissue relative to its upper surface such that the lower surfaces of one or more module absorb heat from the mammalian tissue that is proximate and underlying a given module or such that the lower surfaces of one or more module rejects heat into the mammalian tissue that is proximate and underlying a given module.

77. The device of claim 76, wherein each module of the plurality is positioned relative to the other modules in a predetermined spatial orientation.

78. The device of claim 76, wherein the lower surfaces of the thermoelectric modules absorb heat and the upper surfaces reject heat.

79. The device of claim 76, wherein the lower surfaces of the thermoelectric modules reject heat and the upper surfaces absorb heat.

80. The device of claim 76, further comprising a connecting substrate, wherein at least two thermoelectric modules are fixed to the substrate.

81. The device of claim 80, wherein the fixation to the substrate orients the position of at least two thermoelectric modules relative to each other.

82. The device of claim 80, wherein the connecting substrate comprises a flexible membrane, and wherein the flexible membrane is tailored by addition of chemical constituents, tailoring of stoichiometry, or method of curing such that heat transfer, or other properties such as optical or electrical, may be varied.

83. The device of claim 76, wherein at least two of the thermoelectric modules are selectively operable in a temporal dimension.

84. The device of claim 76, wherein at least two of the thermoelectric modules are selectively operable in a spatial dimension.

85. The device of claim 76, wherein at least two of thermoelectric modules are simultaneously operable in a spatial and temporal dimension.

86. A method for heating, cooling, or maintaining temperature of mammalian tissue, comprising:
a. positioning at least one thermoelectric module in proximity to a mammalian tissue;
b. wherein the module is selectively operable to reject heat from one of the upper or lower surfaces and to absorb heat from one of the upper or lower surfaces, wherein when positioned the lower surface overlies the mammalian tissue and the lower surface is proximate the mammalian tissue relative to the upper surface; and
c. powering the module such that the lower surface absorbs heat from the proximate, underlying mammalian tissue, or such that the lower surface rejects heat into the proximate, underlying mammalian tissue, wherein the absorption of heat acts to cool or maintain the temperature of the mammalian tissue that is proximate and underlying the module and wherein the rejection of heat acts to heat or maintain the temperature of the mammalian tissue that is proximate and underlying the module.

87. The method of claim 86, wherein powering the module comprises initiation of an electric current flow to absorb heat by the lower surface of the thermoelectric module from the mammalian tissue that is proximate and underlying the module and reject heat by the upper surface of the thermoelectric module to cool or maintain the temperature of the mammalian tissue.

88. The method of claim 86, wherein powering the module comprises initiation of the electric current flow to reject heat by the lower surface of the thermoelectric module from the mammalian tissue that is proximate and underlying the module and absorb heat by the upper surface of the thermoelectric module to heat or maintain the temperature of the mammalian tissue.

89. The method of claim 86, further providing a battery power source to operate all of the device components, wherein the device and the power source are portable and capable of operation without requiring connection to a power mains.

90. The method of claim 86, further providing a plurality of thermoelectric modules, wherein each thermoelectric module has an upper and a lower surface, wherein each module is selectively operable to reject heat from one of its upper or lower surfaces and to absorb heat from one of its upper or lower surfaces and wherein the lower surface of each module is positionable in proximity to and overlying mammalian tissue relative to its upper surface such that the lower surfaces of one or more module absorb heat from the mammalian tissue that is proximate and underlying a given module or such that the lower surfaces of one or more module rejects heat into the mammalian tissue that is proximate and underlying a given module.

91. The method of claim 90, further positioning each module of the plurality relative to the other modules in a predetermined spatial orientation.

92. The method of claim 90, further initiating an electric current flow through at least two of the thermoelectric modules to manipulate the temperature of the mammalian tissue that is proximate and underlying each module in a spatial and temporal dimension.

93. The method of claim 90, further initiating an electric current flow through at least two of the thermoelectric modules to independently manipulate the temperature of the mammalian tissue that is proximate and underlying each module in a spatial and temporal dimension.

94. A system for heating, cooling, or maintaining temperature of mammalian tissue comprising:
a. at least one thermoelectric module, the module having an upper and a lower surface, wherein the module is selectively operable to reject heat from one of the upper or lower surfaces and to absorb heat from one of the upper or lower surfaces;
b. wherein the lower surface is positionable in proximity to and overlying mammalian tissue relative to the upper surface such that the lower surface absorbs heat from the proximate, underlying mammalian tissue or such that the lower surface rejects heat into the proximate, underlying mammalian tissue; and
c. a control unit in communication with the thermoelectric module wherein the control unit is used to adjust or maintain application of heat or cooling by the thermoelectric module to the proximate, underlying mammalian tissue.

95. The system of claim 94, wherein the control unit is capable of temporal and spatial modulation of a plurality of thermoelectric modules to affect the temperature of tissue proximate and underlying each module.

96. The system of claim 95, further comprising a thermal sensor associated with each thermoelectric module of the plurality, wherein the thermal sensor provides feedback to the control unit.

97. The system of claim 95, wherein the control unit is programmed to provide both positive and negative heat fluxes across the thermoelectric modules in a checkerboard or similar fashion that may be modulated over time to elicit responses from the body's thermal receptors and therefore impact control and sensation of the affected tissues.

98. The system of claim 95, wherein the control unit controls the thermoelectric modules by individual control calculations based on blood perfusion sensors, or their equivalent such as skin temperature gradient measurements, at the surface of the proximate, underlying mammalian tissue.

99. The system of claim 94, further comprising a connecting substrate, wherein the thermoelectric modules are fixed to the substrate in a predetermined spatial orientation to orient the positions of the thermoelectric modules relative to each other.

100. The system of claim 94, further comprising a battery power source to operate all of the system components, wherein the battery power source is portable and capable of operation without requiring connection to a power mains.
